(19) United States
(12) Patent Application Publication
(10) Pub. No.: US 2007/0087976 A1
(43) Pub. Date: Apr. 19, 2007

(54) Topiramate and pharmaceutical formulations thereof

(76) Inventors: Olaf Generlich, Hamburg (DE); Klaus Glanzer, Hamburg (DE)

Correspondence Address: SMITH PATENT CONSULTING CONSULTING, LLC, 3309 DUKE STREET, ALEXANDRIA, VA, 22314, US

(21) Appl. No.: 11/392,752

(22) Filed: Mar. 30, 2006

(30) Foreign Application Priority Data
Oct. 17, 2005 (DE) DE202005016250.7

(51) Int. Cl. A61K 31/7008

(52) U.S. Cl. 514/23

(57) ABSTRACT

As demonstrated herein, topiramate can be rendered substantially non-hygroscopic if present in a particularly pure form and with a small particle size. Accordingly, the present invention relates to a highly pure form of topiramate, having a particle size of ≦250 μm, and pharmaceutical formulations which contain this active substance.

The present invention relates to topiramate and pharmaceutical formulations which contain this active substance.

Topiramate (2,3:4,5-bis-O-(1-methylethylidene)-β-D-fructopyranose sulfamate) of the general formula I: 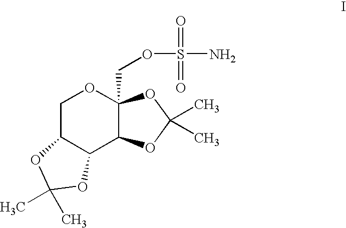
is a known antiepileptic agent. The compound, which belongs to the class consisting of the sulfamoyl-substituted saccharides, is disclosed in EP-A-0 138 441.

Processes for the synthesis of topiramate are described, for example, in EP-A-0 533 483, WO 03/097656 and the still unpublished European Patent Application No. 04.019684.2.

Topiramate is commercially available in the form of tablets, which are available, inter alia, under the trade name Topamax for oral administration with 25 mg, 50 mg, 100 mg or 200 mg of active substance. WO 98/00130 discloses that these tablets contain anhydrous lactose, pregelatinized starch, microcrystalline cellulose, sodium starch glycolate, magnesium stearate, purified water, carnauba wax, hydroxypropylmethylcellulose, titanium dioxide, polyethylene glycol, synthetic iron oxide and polysorbate 80 as inactive constituents.

WO 01/89445 states that topiramate tablets are unstable in the presence of moisture and heat, which leads to visible brown to black discolorations and to the formation of sulfate ions. The greater the amount of free water which is present in the tablets the more rapid is the occurrence of degradation reactions which manifest themselves through color changes. In addition, Topamax tablets absorb moisture very rapidly. For example, according to WO 01/89445 the tablet weight increases by about 1.2% at 60% relative humidity (r.h.) after two hours.

This stability problem of topiramate tablets was originally solved by packing the tablets in HPDE bottles which were provided with a drying agent. Alternatively, it was proposed to offer the tablets in blister packs which were likewise to be provided with a drying agent. In order to avoid the presence of a drying agent, WO 01/89445 proposes drying the topiramate tablets to a residual free water content of from 0.4% to 1.4% before packing in blister packs.

There is furthermore a need for pharmaceutical formulations for topiramate which are stable before packing, even without complicated packs and pretreatments. An object of the present invention is therefore to provide topiramate in a form which has a long shelf-life even during prolonged storage and even at elevated temperatures and is therefore particularly suitable for the preparation of pharmaceutical formulations.

It has now surprisingly been found that topiramate is virtually no longer hygroscopic if it is present in particularly pure form and with a small particle size. The present invention therefore relates to topiramate which is present in highly pure form and in which at least 90% of the particles have a particle size of ≦250 μm.

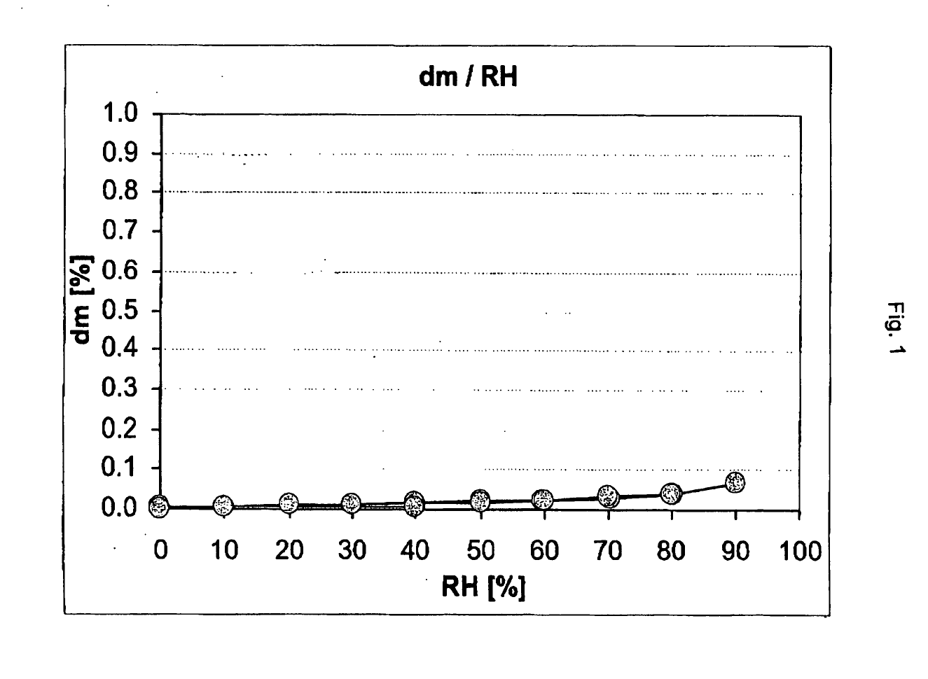

It has been found that highly pure topiramate which is present in said particle size is substantially non hygroscopic. Thus, the pure active substance absorbs less than 0.1% of water at 25° C. at 90% r.h., as indicated by the sorption isotherm shown in FIG. 1. Storage even at 80° C. for one month leads to no color changes. The topiramate according to the invention is therefore particularly suitable for the preparation of pharmaceutical formulations.

Topiramate in the highly pure form is understood here as meaning an active substance which contains at least 99% by weight of topiramate. Preferably, the active substance contains not more than 0.5% by weight, in particular not more than 0.2% by weight, of impurities, water not being counted as an impurity.

According to the invention, the particle size of the active substance is chosen so that at least 90% of the particles have a particle size of ≦250 μm. Preferably, at least 90% of the particles have a particle size of ≦200 μm. The mean particle size should be ≦150 μm, preferably ≦120 μm and particularly preferably in the range of 30-80 μm. Suitable methods for establishing the particle size are known to the person skilled in the art and can easily be chosen by him on the basis of his technical knowledge. Customary methods are described in W. A. Ritschel, Die Tablette [The tablet], 2nd, edition, chapter 3, Mischen und Zerkleinern [Mixing and comminuting], Editio Cantor Verlag Aulendorf 2002. The desired particle size can be established, for example, by classification, e.g. milling and/or sieving.

Owing to the low water absorption, the topiramate according to the invention is suitable for the preparation of pharmaceutical formulations, in particular In the form of tablets or capsules. The present invention therefore also relates to pharmaceutical formulations which contain topiramate according to the present description. In a particularly preferred embodiment, the pharmaceutical formulation is obtainable by direct compression, and the tablets concerned are in particular directly compressed tablets.

For the preparation of the tablets, the active substance is mixed with customary excipients known to the person skilled in the art and preferably directly compressed. Suitable excipients are, for example, microcrystalline cellulose, magnesium stearate, lactose monohydrate, cornstarch, crospovidone and colloidal silica. The amounts of the individual constituents can be chosen by the person skilled in the art so that tablets having a desired content of active substance are obtained. Tablets can be coated in a suitable manner, polymethacrylate and hydroxypropylmethylcellulose-containing films being particularly preferred.

The tablet formulations according to the invention which are obtainable by direct compression absorb less moisture than commercially available tablets. It has been found that Topamax 200 mg or Epitomax 100 mg absorb 9% of water at 25° C. and 90% r.h. In contrast, topiramate tablets according to the invention, as in example 1 below, absorb only 5.5% of water under the same conditions.

Furthermore, with the following exemplary tablet formulations according to the invention, it has been found that, contrary to the teaching of WO 01/89445, topiramate tablets having a free water content of about 2% (examples 1 and 3) to 3% (example 2), i.e. without predrying to less than 1.4% of free water, have none of the instabilities described in the WO document, such as discoloration and formation of sulfate ions, even on open storage in the unpacked state for two months at 40° C. and 75% r.h.

The present invention is now explained in more detail with reference to the following examples, which are not to be interpreted as being limiting.

### EXAMPLES

The formulations stated in the following examples are suitable for the manufacture of tablet cores by direct compression. The tablet cores can be coated with a suitable film. The stated percentages are % by weight.

### Example 1

| | | |
|---|---|---|
| Topiramate | 57.15% | |
| Microcrystalline cellulose | 42.35% | |
| Magnesium stearate | 0.50% | |
| Weight | approx. 350 | mg |
| Hardness | 75 | N |
| Disintegration time | 15 | s |

### Example 2

| | | |
|---|---|---|
| Topiramate | 41.70% | |
| Microcrystalline cellulose | 57.80% | |
| Magnesium stearate | 0.50% | |
| Weight | approx. 480 | mg |
| Hardness | 160 | N |
| Disintegration time | 15 | s |

### Example 3

| | |
|---|---|
| Topiramate | 41.50% |
| Microcrystalline cellulose | 16.50% |
| Lactose monohydrate/cornstarch (85:15) | 35.50% |
| Crospovidone | 5.00% |
| Colloidal silica | 1.00% |
| Magnesium stearate | 0.50% |

Two batches were produced with the following properties:

| a.) | | |
|---|---|---|
| Weight | approx. 120 | mg |
| Hardness | 65 | N |
| Disintegration time | 10 | s |
| b.) | | |
| Weight | approx. 480 | mg |
| Hardness | 190 | N |
| Disintegration time | 39 | s |

The tablets produced according to the above examples had a free water content of about 2% (examples 1 and 3) or 3% (example 2). Even on open storage in the unpacked state for two months at 40° C. and 75% r.h., they showed no instabilities in respect of discoloration or formation of sulfate ions.

1. Topiramate, which is present in highly pure form and in which at least 90% of the particles have a particle size of ≦250 μm.

2. The topiramate of claim 1, which contains not more than 0.5% by weight of impurities.

3. The topiramate of claim 2, which contains not more than 0.2% by weight of impurities.

4. The topiramate of claim 1, wherein at least 90% of the particles have a particle size of ≦200 μm.

5. The topiramate of claim 1, having a mean particle size of ≦150 μm.

6. Topiramate, which is substantially non hygroscopic.

7. The topiramate of claim 1, which absorbs <0.1% of water, based on the weight of the topiramate before the water absorption, at 25° C. and 90% relative humidity.

8. The topiramate of claim 1, which shows no color changes after storage for at least one month at 80° C.

9. A pharmaceutical formulation comprising the topiramate of claim 1.

10. The pharmaceutical formulation of claim 9, which is present in the form of a tablet or a capsule.

11. The pharmaceutical formulation of claim 9, obtained by direct compression.

12. The topiramate of claim 1, having a mean particle size of ≦120 μm.

13. The topiramate of claim 1, having a mean particle size ranging from 30-80 μm.
